(19) United States
(12) Patent Application Publication
(10) Pub. No.: US 2024/0041347 A1
(43) Pub. Date: Feb. 8, 2024

(54) DETERMINATION DEVICE, MEDICAL APPARATUS, ADJUSTMENT UNIT, COMPUTER PROGRAM PRODUCT, STORAGE MEANS AND METHOD FOR DETERMINING A CARBON DIOXIDE CONCENTRATION IN MEASUREMENT GAS

(71) Applicant: Drägerwerk AG & Co. KGaA, Lübeck (DE)

(72) Inventor: Hans-Ullrich HANSMANN, Lübeck (DE)

(21) Appl. No.: 18/011,578

(22) PCT Filed: Jun. 28, 2021
(86) PCT No.: PCT/EP2021/067623

§ 371 Date: Dec. 20, 2022

(30) Foreign Application Priority Data
Jul. 3, 2020 (DE) 10 2020 117 607.4

(51) Int. Cl. A61B 5/083; A61B 5/097; A61B 5/08; A61B 5/087

(52) U.S. Cl. CPC A61B 5/0836; A61B 5/097; A61B 5/082; A61B 5/087

(57) ABSTRACT

A process for determining a carbon dioxide concentration in measured gas, includes branching off the gas from a main line (15) of a medical device (12) through a branch line (14) to a sensor unit (11) during an inhalation phase and an exhalation phase of a person (13). The gas is delivered by a fluid delivery unit that is adaptively set taking an airway pressure in the main line into consideration for generating a uniform volume flow and/or gas pressure of the measured gas in the branch line to the sensor unit during the inhalation phase and the exhalation phase. The concentration of carbon dioxide in the measured gas is determined by the sensor unit. A determination device (10), the medical device, a setting unit (26), a computer program product (29) and a storage device (30) with the program may each be provided to carry out the process.

### CROSS REFERENCE TO RELATED APPLICATIONS

This application is a United States National Phase Application of International Application PCT/EP2021/067623, filed Jun. 28, 2021, and claims the benefit of priority under 35 U.S.C. § 119 of German Application 10 2020 117 607.4, filed Jul. 3, 2020, the entire contents of which are incorporated herein by reference.

### TECHNICAL FIELD

The present invention pertains to a process for determining a concentration of carbon dioxide in measured gas, especially from and/or in a medical device. The present invention further pertains to a computer program product, which comprises commands to carry out such a process, and to a storage device, on which such a computer program product is stored. The present invention further pertains to a determination device, to a medical device and to a setting unit for determining the carbon dioxide concentration.

### BACKGROUND

Carbon dioxide is one of the most important parameters for assessing the ventilation efficiency during the ventilation of a person by a ventilator. A precise and reliable monitoring of the carbon dioxide concentration is therefore of vital importance during the ventilation.

Various physical and/or chemical methods come into consideration for determining the carbon dioxide concentration. For example, the carbon dioxide concentration can be detected by means of infrared sensors, electrochemical sensors, a colorimetric method, or even by means of mass spectrometers. Some of these methods have a complex measuring set-up, are correspondingly expensive as a result and/or are not suitable for a continuous detection of the carbon dioxide concentration.

Furthermore, a system is known, in which the carbon dioxide concentration in the measured gas can be inferred by means of the heat conduction of a measured gas or of a gas sample at a sensor unit. For example, inhalation gas as well as exhalation gas are admitted to a sensor unit by means of diffusion for the determination of the carbon dioxide concentration at a short distance from a so-called mainstream or a main line. Such a system is known from the German patent application DE 10 2010 047 159 A1. Furthermore, a hydrophobic barrier against condensing moisture is proposed there. It is problematic in this system that cross influences acting via gas parameters, such as the measured gas temperature and/or the moisture content of the measured gas synchronously with the breathing phases, i.e., inhalation and exhalation, lead to an insufficiently accurate determination of the carbon dioxide concentration in the measured gas based on the lack of selectivity in the sensor unit. In other words, the fluctuating moisture content due to inhalation and exhalation leads to a fluctuating moisture level at the sensor depending on the coating of the hydrophobic barrier. This may lead to changed measured values and to a corresponding measuring inaccuracy as well as to a partial to complete gas barrier, due to which the desired measurement cannot be continued.

Moreover, measuring systems are known, in which measured gas is suctioned off or branched off to a suitable sensor unit by means of a pump via a branch line from the main line to determine the carbon dioxide concentration by means of the sensor unit. The drawback of the prior-art solutions of this type is that the volume flow of the measured gas in the branch line generated by the pump leads to an influence on the measured signal. A pressure difference develops over the branch line due to the changing airway pressure at the patient. As a result, a changing volume flow develops, which generates a measured signal that is synchronous with the breathing phases. As a result, it is hardly possible to determine with sufficient accuracy the carbon dioxide difference between the two breathing phases necessary for the determination of the carbon dioxide determination. In addition, the correct connection of both phases over time is disturbed by the changing volume flow.

### SUMMARY

An object of the present invention is to take the above-described problems at least partially into consideration. In particular, the object of the present invention is to create a device and a process for the simplest possible, cost-effective and accurate determination of the concentration of carbon dioxide in measured gas from a medical device of this type.

The above object is accomplished by features according to the invention. In particular, the above object is accomplished by a process with features according to the invention, by a determination device with features according to the invention, by a medical device with features according to the invention, by a setting unit with features according to the invention, by a computer program product with features according to the invention as well as by a storage device with features according to the invention. Further advantages of the present invention appear from this disclosure including from the description and from the figures. Features that are described in connection with the process are, of course, also valid in connection with the determination device according to the present invention, with the medical device according to the present invention, with the setting unit according to the present invention, with the computer program product according to the present invention, with the storage device according to the present invention, and also vice versa, so that reference is and/or can mutually always be made to the individual aspects of the present invention concerning the disclosure.

According to a first aspect of the present invention, a process is provided for determining a carbon dioxide concentration in measured gas. The process has the following steps:
branching off the measured gas from a main line of a medical device through a branch line to a sensor unit during an inhalation phase of a person who is connected to the medical device as inhalation gas and during an exhalation phase of the person as exhalation gas,
delivering the measured gas from the main line through the branch line to the sensor unit by means of a fluid delivery unit,
adaptively setting the fluid delivery unit taking into consideration (based on) an airway pressure in the main line for generating a uniform volume flow and/or gas pressure of the measured gas in the branch line to the sensor unit during the inhalation phase and during the exhalation phase, and
determining the concentration of carbon dioxide in the measured gas by means of the sensor unit.

Accordingly, it is proposed to actuate the fluid delivery unit, for example, in the form of a suction pump, with the previous knowledge of the airway pressure such that the volume flow remains as constant as possible during the different breathing phases or during the inhalation phase, and in particular during the inhalation phase of a ventilation by the medical device, and during the exhalation phases, especially during the exhalation phase of a ventilation by the medical device, or such that the changes in the volume flow during the different breathing phases are or become at least as small as possible compared to the carbon dioxide concentration changes between the carbon dioxide concentration in the exhalation phase and the carbon dioxide-free or essentially carbon dioxide-free inhalation phase.

Branching off of the measured gas from the main line of the medical device during the inhalation phase and during the exhalation phase is defined herein as the measured gas being continuously branched off from the beginning of the inhalation phase up to the end of the exhalation phase and subsequently especially without interruption, in principle, any number of times again from the beginning of an additional inhalation phase up to the end of an additional exhalation phase.

The fact that the fluid delivery unit is adaptively set for generating a uniform volume flow and/or gas pressure of the measured gas in the branch line to the sensor unit during the inhalation phase and during the exhalation phase can correspondingly be defined as the fluid delivery unit being adaptively set taking into consideration the current airway pressure in the main line such that a volume flow and/or a gas pressure of the measured gas in the branch line to the sensor unit is generated, which volume flow and/or gas pressure continuously remain the same to the extent possible or have only slight changes to the extent possible from the inhalation phase over the exhalation phase and additional possible, subsequent inhalation phases and exhalation phases. The inhalation phase of the person may be defined as an inhalation process by the person with or without at least partial assistance by the medical device. The exhalation phase of the person may be defined as an exhalation process by the person with or without at least partial assistance by the medical device.

It was first discovered within the framework of the present invention that with a simple measuring set-up with the use of the branch line, the sensor unit and the fluid delivery unit, relatively small volume flows in the branch line are, in principle, already sufficient for the desired determination of the carbon dioxide concentration. As a result, suction volume flows of 100 mL/min to 200 mL/min are used or set, as a rule, for so-called suctioning carbon dioxide measurements in the measured gas. Such volume flows are necessary because of the components used, such as filters, hoses and dehumidifiers. It was now discovered here that simple suction units, which are configured as single-use units, make do not only with markedly smaller volume flows in a range of, for example, 40 mL/min to 60 mL/min as well as with relatively low gas pressures in a range of, for example, 25 mbar to 35 mbar, but also still sufficiently deliver measured gas in the process to the sensor unit for the desired determination of the carbon dioxide concentration in the measured gas.

However, a relatively high dependence on the airway pressures of the patient and/or of the medical device arise due to the low gas pressure and/or due to the small volume flow. The volume flow and the gas pressures during the suctioning vary with the gas pressures in the breathing phases of the patient being ventilated by the medical device and/or of the medical device. To prevent or at least to reduce this effect, the fluid delivery unit is actuated with the previous knowledge of the airway pressure or a corresponding patient pressure such that the volume flow remains as constant as possible in the branch line and/or the gas pressure remains as constant as possible in the branch line. An additional volume flow measurement shall be dispensed with here to the extent possible.

The airway pressure changes from, for example, continuously between about 25 mbar during the inhalation phase and, for example, about 5 mbar during the exhalation phase during the usual ventilation of a patient. If measured gas shall now be delivered at, for example, 50 mL/min through the branch line to the sensor unit, only a minimal assistance by the fluid delivery unit is needed during the inhalation phase.

During the exhalation phase, by contrast, a markedly higher suction output is necessary, since the pressure difference is only ⅕ compared to the surrounding area. About 30 mbar are necessary for a gas flow of, for example, 50 mL/min. If the patient has an airway pressure of, for example, 25 mbar and/or such an airway pressure is at least partially set by the medical device, the fluid delivery unit has to additionally generate only 5 mbar during the inhalation phase to achieve the desired volume flow. An additional 25 mbar are needed by the fluid delivery unit to achieve the same volume flow during the exhalation phase with, for example, only 5 mbar airway pressure.

The fluid delivery unit, especially a pump vacuum of a fluid delivery unit configured as a pump, can now be set such that the sum of the airway pressure and the pump pressure or the pressure generated by the fluid delivery unit together is 30 mbar.

Such a process is shown in the table below.

| | | | | | Sum of | |
|---|---|---|---|---|---|---|
| | | Time of | | | pressure | Volume |
| | | phase | | Suction | over | flow in |
| | Duration | beginning | PAW | pressure | branch | branch |
| Phase | [sec] | [sec] | [mbar] | [mbar] | line | line |
| Beginning | 0.3 | T | 5 → 25 | 25 → 5 | 30 | 50 |
| of | | | | | | |
| inhalation | | | | | | |
| Inhalation | 2.7 | T + 0.3 | 25 | 5 | 30 | 50 |
| Beginning | 0.3 | T + 3 | 25 → 5 | 5 → 25 | 30 | 50 |
| of | | | | | | |
| exhalation | | | | | | |
| Exhalation | 2.7 | T + 3.3 | 5 | 25 | 30 | 50 |
| Beginning | 0.3 | T + 6 | 5 → 25 | 25 → 5 | 30 | 50 |
| of | | | | | | |
| additional | | | | | | |
| inhalation | | | | | | |

In prior-art, suctioning determination devices, relatively high pressure drops are generated because of the filters, water traps, branch lines used and considerably higher gas flows or volume flows of, for example, 200 mL/min compared to the proposed determination device and/or measuring unit, so that the pressure difference for the output of the fluid delivery unit is also in a markedly higher range of, for example, 150 mbar to 200 mbar. The additional pressure difference is subordinate to the airway pressure here, and the fluid delivery unit, especially in the form of a pump, can run with a constant output and generate an average gas flow. In case of such systems, no considerations had to be given hitherto to an actuation of the fluid delivery unit according to the present invention or to the adaptive setting of the fluid delivery unit taking the airway pressure into consideration.

Especially the heat conductivity of the exhalation gas is measured in the sensor unit to determine a carbon dioxide concentration in the measured gas. The measurement is carried out by means of a micro structured heating element on a thin membrane of the sensor unit. A thermophilic unit, which measures an excess temperature of the gas close to the heating element in reference to a silicone frame of the membrane, is located next to the heating element. The determination of the carbon dioxide concentration by means of the heat conductivity of the measured gas is known, in principle, in the state of the art. Further details can be found in, for example, the German patent application DE 10 2010 047 159 A1. The importance of a constant volume flow of the measured gas through the branch line for determination of the carbon dioxide concentration in the measured gas was, however, discovered within the framework of the present invention, taking into consideration the heat conductivity of the measured gas, and the proposed process was then developed. Consequently, the sensor unit preferably comprises a heat conduction sensor for measuring the heat conductivity and/or the heat conduction of the measured gas, i.e., the heat conductivity of the branched-off part of the inhalation gas as well as of the branched-off part of the exhalation gas. The carbon dioxide concentration is subsequently determined on the basis of the measured heat conductivity. The fact that the carbon dioxide concentration is determined by means of the sensor unit may consequently be defined as the carbon dioxide concentration being determined on the basis of different measurements and calculations and the sensor unit being used in this case, or in order words, as the carbon dioxide concentration in the measured gas being determined on the basis of a heat conductivity of the measured gas, which heat conductivity was measured by the sensor unit. The carbon dioxide differences can be determined by means of the sensor unit and the carbon dioxide concentration is calculated on the basis of the measured values and/or determined on the basis of, for example, a look-up table. As was already mentioned above, the measured gas therefore comprises preferably inhalation gas and exhalation gas. Accordingly, the relative carbon dioxide concentration in the exhalation gas can be determined by the carbon dioxide difference between the inhalation gas and the exhalation gas.

The process for determining the carbon dioxide concentration is especially carried out as a process for determining the carbon dioxide concentration during a ventilation by the medical device of the person connected to the medical device and/or at least during an operation of the medical device. Accordingly, the adaptive setting of the fluid delivery unit can be at least partially carried out without current information about the present airway pressure, for example, in the form of an emergency program and/or a transition program. The process may be carried out as a process for determining the carbon dioxide concentration in measured gas from a main line or from the so-called mainstream of a medical device, especially of a ventilator. The adaptive setting of the fluid delivery unit can especially be defined as a continuous setting and/or adjustment of the fluid delivery unit as a function of the current airway pressure in the main line. In other words, the fluid delivery unit is set and/or actuated differently as a function of the current airway pressure in the main line.

The adaptive setting of the fluid delivery unit is especially carried out by means of a suitable setting unit, for example, in the form of a control device of the medical device. The measured gas may be defined as a gas sample to be measured at the sensor unit. The measured gas corresponds during the inhalation phase to a part of the inhalation gas and during the exhalation phase to a part of the exhalation gas. In other words, a part of the inhalation gas is branched off as measured gas during the inhalation phase and a part of the exhalation gas is likewise branched off as measured gas during the exhalation phase.

The delivery of the measured gas may be defined as a suctioning off of the measured gas from the main line into the branch line to the sensor unit. The fluid delivery unit is configured in this case especially as a pump and/or suction pump. The process steps described do not have to be carried out in the order indicated. Rather, the process steps may be carried out simultaneously at least partially. A uniform volume flow may be defined in the present case as a volume flow, the value of which changes over time from at least one inhalation phase and an exhalation phase subsequent thereto by less than 20%, especially by less than 10%.

The airway pressure corresponds to the gas pressure of the inhalation gas in the main line during the inhalation phase and to the gas pressure of the exhalation gas in the main line during the exhalation phase. The airway pressure may consequently also be defined as the gas pressure, and in particular the gas pressure of the inhalation gas and the gas pressure of the exhalation gas, in the main line. The airway pressure may be measured as the actual airway pressure and/or determined or used as a set airway pressure.

According to one embodiment of the present invention, it is possible that the airway pressure in the main line is measured in one process by means of an airway pressure sensor and the fluid delivery unit is adaptively set for generating a uniform volume flow and/or gas pressure of the measured gas in the branch line to the sensor unit, with the use of the measured airway pressure in the main line. In other words, the delivery unit is set with the use of the airway pressure actually measured. Consequently, the carbon dioxide concentration in the measured gas can be especially accurately determined. The main line may have here an inhalation gas line section for sending the inhalation gas, especially only the inhalation gas, and a total gas line section for sending the inhalation gas as well as the exhalation gas, wherein the airway pressure is measured in and/or at the total gas line section.

Further, a process is proposed within the framework of the present invention for determining the concentration of carbon dioxide in the measured gas during a pressure-controlled ventilation of the person by the medical device, wherein the fluid delivery unit is adaptively set for generating a uniform volume flow and/or a uniform gas pressure of the measured gas in the branch line to the sensor unit, with the use of an airway pressure in the main line, which airway pressure was set by the pressure-controlled ventilation of the person. An airway pressure sensor as was mentioned above can be dispensed with due to the pressure-controlled ventilation of the person. The airway pressure needed for the adaptive setting of the fluid delivery unit may be read out and used, for example, directly from a control device of the medical device. The fluid delivery unit can accordingly be adaptively set with the use of the airway pressure for the pressure-controlled ventilation, which airway pressure was set for the operation of the medical device. Further, it is possible that in a process according to the present invention for determining the carbon dioxide concentration in the measured gas during a volume-controlled ventilation of the person by means of the medical device, the fluid delivery unit is adaptively set for generating a uniform volume flow and/or gas pressure of the measured gas in the branch line to the sensor unit, with the use of an airway pressure in the main line, which airway pressure results from the volume-controlled ventilation of the person.

Furthermore, it is possible that in a process according to the present invention, the fluid delivery unit has a piezo pump for delivering the measured gas from the main line through the branch line to the sensor unit and an operating voltage of the piezo pump is adaptively set for generating a uniform volume flow and/or gas pressure of the measured gas in the branch line to the sensor unit, taking into consideration the airway pressure as well as with the use of a look-up table. With the use of the piezo pump and an associated look-up table, especially a characteristic diagram of this kind with characteristic curves, the fluid delivery unit can be set in the desired manner rapidly, simply and with cost-effective means. Based on the characteristic curves of the look-up table, it is possible to rapidly detect and/or determine what volume flow and what gas pressure is present or will be present in the branch line at what voltage or operating voltage of the piezo pump. However, the necessary preliminary consideration for using the piezo pump and an associated look-up table was that experiments carried out within the framework of the present invention showed that the low pressures and small volume flows mentioned above are already sufficient for the desired determination of the carbon dioxide concentration by means of measuring heat conduction in the measured gas. A piezo pump would be less suitable or not at all suitable for conventional systems with volume flows of the measured gas of, for example, more than 100 mL/min and/or more than 40 mbar.

In addition, it is possible that in a process according to the present invention, the fluid delivery unit is adaptively set for generating a uniform volume flow and/or gas pressure in the measured gas in the branch line to the sensor unit, only taking into consideration an airway pressure during an inhalation phase in the main line or only taking into consideration an airway pressure during an exhalation phase of the ventilation in the main line. Experiments carried out within the framework of the present invention showed that the consideration of, for example, only the inhalation phase or the exhalation phase is nevertheless sufficient to determine the carbon dioxide concentration with sufficient accuracy with reduced computing power and/or to generate the as uniform as possible volume flow and/or as uniform as possible gas pressure of the measured gas.

Moreover, it is possible that in a process according to the present invention, the fluid delivery unit is operated with consistent output during an exhalation phase of the ventilation for generating a uniform volume flow and/or gas pressure of the measured gas in the branch line to the sensor unit and is adaptively set during an inhalation phase of the ventilation taking into consideration the airway pressure during the inhalation phase of the ventilation. Consequently, the fluid delivery unit does not have to be adaptively set and/or operated continuously. Also, a necessary computing power for determining the carbon dioxide concentration can thus be reduced and the process can be carried out in a resource-saving manner. The fluid delivery unit is in this case operated during the exhalation phase of the ventilation with consistent output, especially with maximum output or with a consistent output in a range of 80% to 100% of the maximum output of the fluid delivery unit.

In another embodiment variant of the present invention, it is possible that in one process the fluid delivery unit is adaptively set or operated for generating a uniform volume flow and/or gas pressure of the measured gas in the branch line to the sensor unit during an exhalation phase of the ventilation taking into consideration the airway pressure during the exhalation phase of the ventilation and is deactivated during an inhalation phase of the ventilation. The process can thereby be operated efficiently and especially in an energy-saving manner. Moreover, the lifetime of the fluid delivery unit can thus be extended. The fact that the fluid delivery unit is deactivated may be defined as the fluid delivery unit being or becoming switched off and/or at least not being operated.

According to another aspect of the present invention, a determination device is provided for determining a carbon dioxide concentration in measured gas from a medical device during a ventilation of a person by means of the medical device. The determination device comprises:
a sensor unit for determining the concentration of carbon dioxide in the measured gas,
a branch line for branching off the measured gas from a main line of the medical device during an inhalation phase of the person as inhalation gas and during an exhalation phase of the person as exhalation gas to the sensor unit,
a fluid delivery unit for delivering the measured gas from the main line through the branch line to the sensor unit, and
a setting unit, which is configured and embodied for the adaptive setting of the fluid delivery unit taking into consideration an airway pressure in the main line for generating a uniform volume flow and/or gas pressure of the measured gas in the branch line to the sensor unit during the inhalation phase and during the exhalation phase.

The determination device according to the present invention thus offers the same advantages as they have been described in detail with reference to the process according to the present invention. The determination device may be configured as a device separate from the medical device or as a component of the medical device. The setting unit may comprise a control device of this kind of the medical device or may be configured as a component of such a control device.

According to another embodiment of the present invention, a determination device may have an airway pressure sensor for measuring the airway pressure in the main line, wherein the setting unit is configured and embodied for the adaptive setting of the fluid delivery unit taking into consideration and/or using the measured airway pressure in the main line for generating a uniform volume flow and/or gas pressure of the measured gas in the branch line to the sensor unit.

The determination device may further be configured for determining the concentration of carbon dioxide in the measured gas during a pressure-controlled ventilation of the person by the medical device, wherein the setting unit may be configured and embodied for the adaptive setting of the fluid delivery unit for generating a uniform volume flow and/or gas pressure of the measured gas in the branch line, with the use of an airway pressure in the main line, which airway pressure was set by the pressure-controlled ventilation of the person.

Moreover, the determination unit can be configured for determining the concentration of carbon dioxide in the measured gas during a volume-controlled ventilation of the person by the medical device, wherein the setting unit may be configured and embodied for the adaptive setting of the fluid delivery unit for generating a uniform volume flow and/or gas pressure of the measured gas in the branch line to the sensor unit, with the use of an airway pressure in the main line, which airway pressure results from the volume-controlled ventilation of the person.

In addition, the fluid delivery unit may have a piezo pump for delivering the measured gas from the main line through the branch line to the sensor unit, wherein the setting unit is configured for the adaptive setting of an operating voltage of the piezo pump in order to generate the uniform volume flow and/or gas pressure of the measured gas in the branch line to the sensor unit, taking into consideration the airway pressure as well as with the use of a look-up table.

In case of a determination device according to the present invention, the setting unit may further be configured and embodied for the adaptive setting of the fluid delivery unit and for generating a uniform volume flow and/or gas pressure of the measured gas in the branch line to the sensor unit, only taking into consideration an airway pressure during an inhalation phase of the ventilation in the main line or only taking into consideration an airway pressure during an exhalation phase of the ventilation in the main line.

In addition, the setting unit of a determination device according to the present invention may be configured and embodied to operate the fluid delivery unit with consistent output for generating a uniform volume flow and/or gas pressure of the measured gas in the branch line to the sensor unit during an exhalation phase of the ventilation and to adaptively set the fluid delivery unit during an inhalation phase of the ventilation taking into consideration the airway pressure during the inhalation phase of the ventilation. In addition, the setting unit may be configured and embodied to adaptively set the fluid delivery unit for generating a uniform volume flow and/or gas pressure of the measured gas in the branch line to the sensor unit during an exhalation phase of the ventilation taking into consideration the airway pressure during the exhalation phase of the ventilation and to deactivate the fluid delivery unit during an inhalation phase of the ventilation.

A determination device according to the present invention may further have at least one heat and moisture exchanger (also known as a heat and moisture exchanger filter) in and/or at the branch line. The setting unit is configured in this case to compensate a possible effect of the heat and moisture exchanger on the volume flow and/or on the gas pressure in the branch line to the extent that, furthermore, a more uniform volume flow and/or a more uniform gas pressure are generated in the branch line. Within the framework of the present invention, it was shown that by using a heat and moisture exchanger for filtering the measured gas, temperature and moisture differences in the measured gas, which are caused during the inhalation phase and during the exhalation phase of the person or of a patient connected to the medical device, can be buffered, compensated, reduced and/or smoothed to the extent that the carbon dioxide concentration can be determined or measured and/or calculated markedly more accurately compared to a system without heat and moisture exchanger. In other words, the heat and moisture exchanger (heat and moisture exchanger filter) contributes to the carbon dioxide concentration being able to be determined even more accurately in a simple manner.

In addition, it was discovered that the heat and moisture exchanger used has no appreciable and/or adverse effect on other gas components to be measured. In other words, the moisture and the heat of the measured gas are distributed uniformly over time, without having an effect on the actually desired effect on the measurement of the differences in heat conduction concerning the presence and the absence of carbon dioxide. Consequently, the heat and moisture exchanger has no effect or essentially no effect on the feed of the quantity of carbon dioxide to the sensor unit. The gas transport is possibly delayed somewhat only by the volume of the heat and moisture exchanger. However, this has no effect or at least no appreciable effect on the desired determination of the carbon dioxide concentration in the measured gas. Changes in heat conduction, which result from changes in temperature and/or moisture level in the measured gas and occur synchronously with the breathing phases, are among the chief causes of inaccurate carbon dioxide measurements in addition to the changed volume flows. This problem can be taken into consideration in a simple, cost-effective and effective manner by using the heat and moisture exchanger.

A heat and moisture exchanger is defined in medical technology as a heat and moisture exchange filter and/or as a filter housing with such a filter material. The heat and moisture exchanger can consequently be defined as a heat and moisture exchanger. Heat and moisture exchangers have hitherto been used especially in a mainstream or in a main line of a ventilator or a corresponding medical device, where inhalation gas and exhalation gas always flow through them alternatingly in the ventilation cycle. Heat and moisture exchangers have hitherto been used especially for an appropriate humidification of the inhalation gas or of the inhaled air of the patient as well as for avoiding cross contamination in the main line. The proposed heat and moisture exchanger of the determination device is configured in terms of its size and/or function preferably for buffering, compensating, reducing and/or smoothing temperature and/or moisture differences of the measured gas branched off for the duration of at least one breath, i.e., including inhalation phase as well as exhalation phase. The heat and moisture exchanger can accordingly be provided not only for the classical filtering of the measured gas, but especially for buffering, compensating, reducing and/or smoothing the temperature and/or moisture differences in the branched-off measured gas. The at least one heat and moisture exchanger may have a filter housing and filter material for filtering the measured gas in the exchanger housing. The filter housing may be configured as a rigid filter housing or as a flexible or elastically deformable filter housing, which has, for example, a tubular configuration. The heat and moisture exchanger may likewise be configured without a filter housing and exclusively as the functionally relevant heat and moisture exchanger filter material, for example, in the form of a hose insert.

Due to the fact that preferably only the measured gas suctioned off flows through the heat and moisture exchanger, i.e., that the total quantity of the gas of the main line does not, in particular, flow through it, the heat and moisture exchanger can have a smaller, especially several times smaller configuration than a conventional heat and moisture exchanger used in the main line. The heat and moisture exchanger is preferably arranged in a measured gas flow direction to the sensor unit upstream of the sensor unit and/or in a state installed in the ventilator upstream of the sensor unit, so that the measured gas can flow through the heat and moisture exchanger before it reaches the sensor unit.

The determination device is preferably configured for use in and/or with a medical device in the form of a ventilator. The branch line preferably has a flexible hose line for sending the branched-off measured gas to the sensor unit. Further, the branch line may be configured in the form of the flexible hose line. In addition, it is possible that the branch line also has, in addition to the hose line, an additional functional component, such as an adapter and/or connection components for connecting the hose line to the main line, to the sensor unit and/or to the heat and moisture exchanger.

The sensor unit may be embodied and/or configured according to a sensor for determining the carbon dioxide concentration in the measured gas, which is described in DE 10 2010 047 159 A1, the measured gas being fed to the sensor unit by means of the fluid delivery unit and especially by means of a suction pump. The branch line has an internal diameter that is smaller, especially several times smaller, than that of a main line of this class for a ventilator.

According to another embodiment of the present invention, it is possible that the at least one heat and moisture exchanger is arranged in the branch line in a determination device. Consequently, the determination device can be made available as an especially compact and correspondingly space-saving determination device. Further, the determination device can be installed at and/or in the medical device in a simple manner. The at least one heat and moisture exchanger may already be arranged in the branch line at the time of the installation. The at least one heat and moisture exchanger is arranged especially within a line volume of the branch line. The branch line may have, for example, a hose line, wherein the at least one heat and moisture exchanger is arranged in at least one part of the inner volume of the hose line. In other words, at least one part of a hose jacket of the hose line can enclose the at least one heat and moisture exchanger over the entire length of the at least one heat and moisture exchanger in a jacket-like manner. The at least one heat and moisture exchanger may quasi be configured in the form of a hose insert. The at least one heat and moisture exchanger is preferably configured in a positive-locking and/or nonpositive manner in the branch line. The outer circumferential surface of the at least one heat and moisture exchanger can accordingly be configured complementarily to an inner circumferential surface of the branch line, especially to an inner circumferential surface of the hose line of the branch line. The external diameter of the at least one heat and moisture exchanger may consequently correspond to the internal diameter at the location of the hose line at which the at least one heat and moisture exchanger is positioned in the hose line, or it may be slightly smaller than the internal diameter at the location of the hose line for the insertion of the at least one heat and moisture exchanger into the branch line.

Further, it is possible that in a determination device according to the present invention the branch line has a main line-side end section for connecting the branch line to the main line and a sensor-side end section for connecting the branch line to the sensor unit, wherein a heat and moisture exchanger is arranged at and/or in the main line-side end section. The one heat and moisture exchanger, especially the only heat and moisture exchanger, is thus arranged as much as possible directly at and/or close to the main line. As a result, the intended buffering or compensation of the temperature and/or moisture differences in the measured gas by the heat and moisture exchanger can be carried out as early as possible upstream of the sensor unit. Undesired condensate in the branch line downstream of the heat and moisture exchanger and/or upstream of the sensor unit can be effectively prevented or at least effectively reduced hereby. This is especially advantageous when the branch line has a longer hose line and critical conditions, for example, cold external temperatures prevail, at which the temperature in the hose line drops markedly below the mask temperature or drops below the dew point of the average humidity. The fact that the sensor-side end section is configured for connecting the branch line to the senor unit can be defined such that a connection junction is formed at the sensor-side end section for the fluid-tight connection of the branch line to the main line, especially at a counter-connection junction of the main line. The fluid-tight connection may be defined as a joining connection through which the measured gas can be sent, especially suctioned, without leakage from the main line into the branch line. The fact that the heat and moisture exchanger is arranged at and/or in the main line-side end section can be defined such that the heat and moisture exchanger is arranged, for example, in the form of a hose insert, at least partially in the main line-side end section of the branch line or of a hose line of the branch line, or that it is arranged as an attached part at least partially outside of such a hose line at the hose line.

Furthermore, it is possible that in a determination device according to the present invention, the branch line has a main line-side end section for connecting the branch line to the main line and a sensor-side end section for connecting the branch line to the sensor unit, the determination device having a first heat and moisture exchanger at and/or in the main line-side end section and a second heat and moisture exchanger at and/or in the sensor-side end section. The sensor unit can be effectively protected from condensing moisture by the second heat and moisture exchanger at and/or in the sensor-side end section. This leads in turn to the feed of measured gas that is free from moisture to the extent possible to the sensor unit and consequently to correspondingly accurate measurement results. The two heat and moisture exchangers are preferably configured, when viewed along the branch line, at spaced locations from one another, for example, by more than 50 cm, especially at spaced locations from one another in a range of cm to 150 cm. The two heat and moisture exchangers preferably have the same size and/or shape.

In addition, it is possible that in a determination device according to the present invention, the first heat and moisture exchanger is configured in the main line-side end section of the branch line in the form of a hose insert, wherein the branch line has, when viewed in a direction of flow of the measured gas through the branch line, a larger internal diameter at the level of the heat and moisture exchanger than it has in an area downstream of the heat and moisture exchanger. Due to the fact that the branch line is less susceptible to condensing moisture in the measured gas downstream of the heat and moisture exchanger, the internal diameter of the branch line can be made relatively small downstream of the heat and moisture exchanger. Material and costs can thus be saved, and the branch line may have a compact configuration. In particular, a dead space in the branch line can be kept relatively small and/or a measurement delay can be kept relatively short hereby. The flow direction of the measured gas through the branch line is viewed in a state of the determination device, in which this determination device is installed in the medical unit. The flow direction thus extends from the main line through the branch line, extending there through the at least one heat and moisture exchanger arranged in and/or at the branch line, and downstream of the least one heat and moisture exchanger to the sensor unit and, moreover, for example, to a pump, which may be arranged downstream of the sensor unit for suctioning the measured gas from the main line into the branch line. The internal diameter at the level of the heat and moisture exchanger is made somewhat larger compared to the internal diameter measured downstream of the heat and moisture exchanger in order to make it possible to accommodate the heat and moisture exchanger with a correspondingly large diameter or external diameter. It is thus possible to comply with the wish to achieve a sufficient buffering effect through the heat and moisture exchanger and to nevertheless ensure a space-saving forwarding of the measured gas to the sensor unit with the shortest delay possible.

In a determination device according to the present invention, the internal diameter of the branch line may have a value in a range of 2 mm to 4 mm at the level of the first heat and moisture exchanger and the internal diameter of the branch line downstream of the first heat and moisture exchanger may have a value in a range of 0.5 mm to 2 mm. It has been shown in comprehensive experiments carried out within the framework of the present invention that possible condensate upstream of the heat and moisture exchanger is relatively unproblematic in case of a diameter in the range of 2 mm to 4 mm. The diameter in a range of 0.5 mm to 2 mm downstream of the heat and moisture exchanger has proved to represent an advantageous compromise concerning a robust branch line and a dead space that is nevertheless as small as possible or a correspondingly short measuring delay. The branch line or hose line may be configured for establishing a flow velocity in a range of 1 m/sec to 1.5 m/sec at a volume flow in a range of 50 mL/min to 70 mL/min.

In a determination device according to the present invention, the at least one heat and moisture exchanger may be arranged, furthermore, in the main line-side end section of the branch line in the form of a hose insert, wherein the branch line, when viewed in the flow direction of the measured gas through the branch line, has a larger internal diameter in an area upstream of the at least one heat and moisture exchanger than downstream of the at least one heat and moisture exchanger. Condensate can thus be prevented from leading to clogging of the branch line upstream of the at least one heat and moisture exchanger and it is possible to achieve downstream of the at least one heat and moisture exchanger the desired compromise concerning a robust branch line and nevertheless a smallest possible dead space or a correspondingly short measuring delay. It proved to be advantageous if the internal diameter of the branch line upstream of the at least one heat and moisture exchanger has a value in a range of 1.5 mm to 4 mm, and the internal diameter of the branch line downstream of the at least one heat and moisture exchanger has a value in a range of 0.5 mm to 2 mm. Advantages can be achieved in terms of a simple manufacture of the branch line if the areas upstream of the heat and moisture exchanger as well as at the level of the heat and moisture exchanger have the same internal diameter. For example, it is thus possible to configure a hose line of the branch line which has an internal diameter having the same value from an area upstream of the heat and moisture exchanger in the sensor-side end section up to an area, in which the heat and moisture exchanger is arranged in the hose line, and which has a smaller internal diameter than upstream of the heat and moisture exchanger or in the area of the heat and moisture exchanger only downstream of the heat and moisture exchanger. The same can be configured analogously concerning an external diameter of such a hose line. In addition, it is possible that the area upstream of the heat and moisture exchanger or the corresponding internal volume of a hose line of the branch line has a smaller internal diameter than in the area of the heat and moisture exchanger, and preferably nevertheless a larger internal diameter than in the area downstream of the heat and moisture exchanger. The internal diameter of an above-described hose line can consequently remain constant over the area upstream of the heat and moisture exchanger up to the area, in which the heat and moisture exchanger is arranged in the hose line, and it can decrease from the area in which the heat and moisture exchanger is arranged in the hose line to the area downstream of the heat and moisture exchanger, or increase from the area upstream of the heat and moisture exchanger to the area, in which the heat and moisture exchanger is arranged in the hose line, and decrease again from the area, in which the heat and moisture exchanger is arranged in the hose line to the area downstream of the heat and moisture exchanger.

In a determination device according to the present invention, the at least one heat and moisture exchanger may further have a length in a range of 8 mm to 20 mm and a width in a range of 2 mm to 6 mm. In particular, the at least one heat and moisture exchanger has a length in a range of 10 mm to 15 mm and a width in a range of 3 mm to 5 mm. To the extent possible, only the measured gas or the suction stream flows according to the present invention through the at least one heat and moisture exchanger, which at least one heat and moisture exchanger can thus be kept relatively small. The prior-art heat and moisture exchangers used hitherto in the main line are configured for patient gas streams of up to 180 L/min. The at least one heat and moisture exchanger according to the present invention is configured for a flow of measured gas in a range of, for example, 30 mL/min to 100 mL/min, and especially in a range of 40 mL/min to mL/min. The branch line can therefore be configured as being a correspondingly small branch line requiring a small amount of material and space as well as in a cost-effective manner. The at least one heat and moisture exchanger is preferably configured as cylindrical and having a length in a range of 8 mm to 20 mm and a diameter in a range of 2 mm to 6 mm.

According to another embodiment variant of the present invention, it is possible that the branch line in a determination device has a hose line with a length in a range of 80 cm to 150 cm. It was found in experiments carried out within the framework of the present invention that an effective buffering effect can already be achieved concerning the desired temperature and/or moisture compensation even in case of such a hose length. The hose line has especially a length in a range of 90 cm to 110 cm. The hose line has the above-mentioned internal diameter in a range of 0.5 mm to 2 mm, preferably over a length of the hose line in a range of 80 cm to 120 cm.

Furthermore, the branch line in a determination device according to the present invention may have a hose line made of silicone or at least partially of silicone. It was shown in experiments carried out within the framework of the present invention that a counter-drying effect, which leads to a further buffering and/or smoothing of fluctuations in the moisture content, is exerted on the measured gas with the use of a silicone hose in the branch line.

It may be additionally advantageous in a determination device according to the present invention if the branch line has a hose line with a PVC coating on an outer circumferential surface of the hose line. Environmental effects on the measured gas, which could lead to an influencing of the measurement results, can be prevented by the PVC coating in a simple and cost-effective manner. The PVC coating preferably has a thickness in a range of 0.1 mm to 0.4 mm.

In a determination device according to another embodiment variant of the present invention, it is possible that the branch line has a Luer lock fitting for establishing a fluid connection to the main line. The branch line can thus be connected or joined in an especially rapid and simple manner to the main line and/or to a connection section of the main line. A counter-Luer lock fitting can thus be arranged at the main line, at the breathing mask and/or at an exhalation valve at the breathing mask of the medical device for a corresponding junction connection between the main line and the branch line, between the breathing mask and the branch line and/or between the exhalation valve and the branch line.

The at least one heat and moisture exchanger may further have a microporous plastic foam in a preferred embodiment of a determination device according to the present invention. The desired compensation effects on the temperature and/or on the moisture in the measured gas can thus be achieved in an especially reliable manner. The at least one heat and moisture exchanger may especially also have an open-pore, salt-coated plastic foam as well. The at least one heat and moisture exchanger can therefore have a moistening efficiency of about 30 mg of water per liter with respect to the inhalation gas.

In a determination device according to the present invention, the fluid delivery unit may further have a piezo pump. As already mentioned above, with the use of the piezo pump, an associated look-up table can be used for a rapid, simple and yet accurate determination of the carbon dioxide concentration and/or for a rapid and reliable generation of an as uniform as possible volume flow and/or of an as uniform as possible gas pressure of the measured gas. A determination device according to the present invention may, in addition, be configured for determining a carbon dioxide concentration in measured gas from a medical device during a pressure-controlled ventilation of a person by means of the medical device, wherein the setting unit is configured for the adaptive setting of the fluid delivery unit with the use of an airway pressure in the main line, which airway pressure was set by the pressure-controlled ventilation of the person. The fact that the setting unit is configured for the adaptive setting of the fluid delivery unit with the use of an airway pressure in the main line, which airway pressure was set by the pressure-controlled ventilation of the person, can be defined such that the setting unit uses the set airway pressure in the main line for the adaptive setting of the fluid delivery unit or adaptively sets the fluid delivery unit based on the set airway pressure.

The determination device may be configured for determining a carbon dioxide concentration in measured gas from a medical device during a volume-controlled ventilation of a person by means of the medical device in another embodiment variant of the present invention, wherein the setting unit is configured for the adaptive setting of the fluid delivery unit with the use of an airway pressure in the main line, which airway pressure results from the volume-controlled ventilation of the person. The fact that the setting unit is configured for the adaptive setting of the fluid delivery unit with the use of an airway pressure in the main line, which airway pressure results from the volume-controlled ventilation of the person, can be defined such that the setting unit uses the airway pressure in the main line, which airway pressure results from the volume-controlled ventilation of the person, for the adaptive setting of the fluid delivery unit and adaptively sets the fluid delivery unit based on the resulting airway pressure

According to another aspect of the present invention, a medical device for ventilating a person is made available. The medical device has a main line for sending inhalation gas and for sending exhalation gas, and a determination device as described in detail above for determining a carbon dioxide concentration in measured gas from the main line. The medical device according to the present invention thus also leads to the advantages described above. The medical device may have a breathing mask and/or an exhalation valve, wherein the main line may be configured for sending inhalation gas to the breathing mask and for sending exhalation gas away from the breathing mask and/or to the exhalation valve. The branch line may be configured for branching off the measured gas from the main line through the breathing mask and/or through the exhalation valve. In case of a medical device according to the present invention, an exhalation valve may accordingly be arranged at the breathing mask, and the main line extends from an exhalation area of the breathing mask to the exhalation valve and from there, i.e., in and/or at the exhalation valve, the branch line is arranged at the main line for branching off the measured gas from the main line. The medical device may have, in addition, a fluid delivery unit, especially a pump, for example, a piezo pump, for delivering, pumping and/or suctioning off the measured gas or inhalation gas and exhalation gas from the main line into the branch line. The medical device and/or the determination device are each configured and embodied for carrying out the process described above.

In a medical device according to the present invention, the main line may have an inhalation gas line section for sending the inhalation gas and a total gas line section for sending the inhalation gas as well as the exhalation gas, wherein the branch line is configured for branching off the measured gas from the total gas line section. In other words, the measured gas can be branched off from a part of the main line, through which both inhalation gas and exhalation gas are sent during the operation of the medical device. The concentration of carbon dioxide in the measured gas is determined or measured especially via a carbon dioxide difference between the inhalation gas and the exhalation gas and is calculated by means of a computing unit of the medical device.

The at least one heat and moisture exchanger may be located within the total gas line section in a medical device according to the present invention. In other words, the branch line is not only connected and/or attached to the main line, but it also extends into the main line; more precisely, into the total gas line section. The heat and moisture exchanger and/or the branch line with a heat and moisture exchanger arranged in it can quasi be arranged and/or guided within the main line. The outer circumferential surface of the branch line can be located at a spaced location from an inner circumferential surface of the main line in an area, in which the heat and moisture exchanger is arranged in and/or at the branch line. As a result, an especially compact and yet functional construction can be achieved. The main line may, furthermore, extend to an exhalation valve of the medical device or through at least a part of the exhalation valve. In this case, the at least one heat and moisture exchanger can also be considered as being arranged within the exhalation valve. This leads to an especially compact and robust construction as well. The heat and moisture exchanger may, in particular, be effectively protected from environmental effects within the main line and/or within the exhalation valve. At least one part of the branch line can extend in a medical device according to the present invention from a position within the main line from the total gas line section into the inhalation gas line section. In other words, the branch line may be guided within the main line or through a main line volume of the main line, which is configured for sending the inhalation gas. In other words, the branch line may be integrated into at least one part of the main line and/or guided in same. The medical device can thus be provided in an especially space-saving manner.

In a medical device according to the present invention, in which an exhalation valve in the total gas line section is configured for releasing exhalation gas from the medical device into the area surrounding the medical device, the at least one heat and moisture exchanger may be arranged in the exhalation valve. Such an embodiment variant can also be embodied in a relatively compact manner. With a heat and moisture exchanger integrated into the exhalation valve, only the branch line has to be connected to the exhalation valve during the assembly of the medical device and it must then be guided to the sensor unit. The branch line can be replaced when needed in a rapid, simple and cost-effective manner, for example, in the form of a simple hose line. A position within the exhalation valve means that the at least one heat and moisture exchanger and/or a part of the branch line with the at least one heat and moisture exchanger arranged at it and/or in it are arranged in a valve volume of the exhalation valve, through which the exhalation gas as well as the inhalation gas of the main line flow. The branch line is preferably connected to the exhalation valve for branching off the measured gas from the main line. The branch line may have to this end a branch connection and the exhalation valve may have a counter-branch connection to establish a fluid-tight connection to the branch connection.

The medical device described here is preferably made available and/or configured in the form of a ventilator. The medical device can thus be defined as a medical device for ventilating a person, especially a patient. The medical device may also be configured for this purpose in the form of an anesthesia apparatus. The ventilator may preferably be configured and/or embodied in the form of an emergency ventilator, of a ventilator for use in an intensive care unit, of a home ventilator, of a mobile ventilator and/or of a neonatal ventilator.

According to another aspect of the present invention, a setting unit is made available for use in a determination device as described above and/or in a medical device as described above. The setting unit is configured and embodied for the adaptive setting of the fluid delivery unit taking into consideration the airway pressure in the main line, for generating a uniform volume flow and/or gas pressure of the measured gas in the branch line to the sensor unit during the inhalation phase and during the exhalation phase.

In addition, a computer program product, which comprises commands which cause, during the execution of the computer program product by a computer, this computer program product to carry out the process described above, is proposed within the framework of the present invention. The computer program product may be implemented as a computer-readable instruction code in any suitable programming language, for example, in JAVA, C++, C # and/or Python. The computer program product may be stored on a computer-readable storage medium such as a data disk, on a removable drive, on a volatile or non-volatile memory, or on an installed memory/processor (non-transitory computer-readable media). The instruction code may program a computer or other programmable devices such as a control device and/or the setting unit such that the desired functions are executed. Furthermore, the computer program product can become provided and/or may be provided in a network, for example, on the internet, from which it can be downloaded by a user as needed. The computer program product may become embodied and/or may be embodied both by means of a software and by means of one or more special electronic circuits, i.e., in hardware or in any hybrid form, i.e., by means of software components and hardware components. Another aspect of the present invention pertains to a storage device, on which such a computer program product is stored.

Further measures improving the present invention appear from the following description of different exemplary embodiments of the present invention, which are schematically shown in the figures. All the features and/or advantages appearing from the claims, from the description or from the figures, including structural details and arrangements in space, may be essential for the present invention both in themselves and in different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a medical device with a determination device according to a first embodiment of the present invention;

FIG. 2 is a schematic view showing a storage device with a computer program product stored on it;

FIG. 3 is a schematic view showing a medical device with a determination device according to a second embodiment of the present invention;

FIG. 4 is a view showing a flow chart for explaining a process according to a preferred embodiment of the present invention;

FIG. 5 is a view showing characteristic diagrams for showing an airway pressure during a ventilation with uniform volume flow by actuating a fluid delivery unit according to the present invention;

FIG. 6 is a view showing a look-up table for a piezo pump;

FIG. 7 is a view showing characteristic diagrams for explaining an adaptive setting of a fluid delivery unit during a pressure-controlled ventilation by means of a medical device according to the present invention; and

FIG. 8 is a view showing characteristic diagrams for explaining an adaptive setting of a fluid delivery unit during a volume-controlled ventilation by means of a medical device according to the present invention.

### DESCRIPTION OF PREFERRED EMBODIMENTS

Referring to the drawings, elements having the same function and mode of operation are always provided with the same reference numbers in the figures.

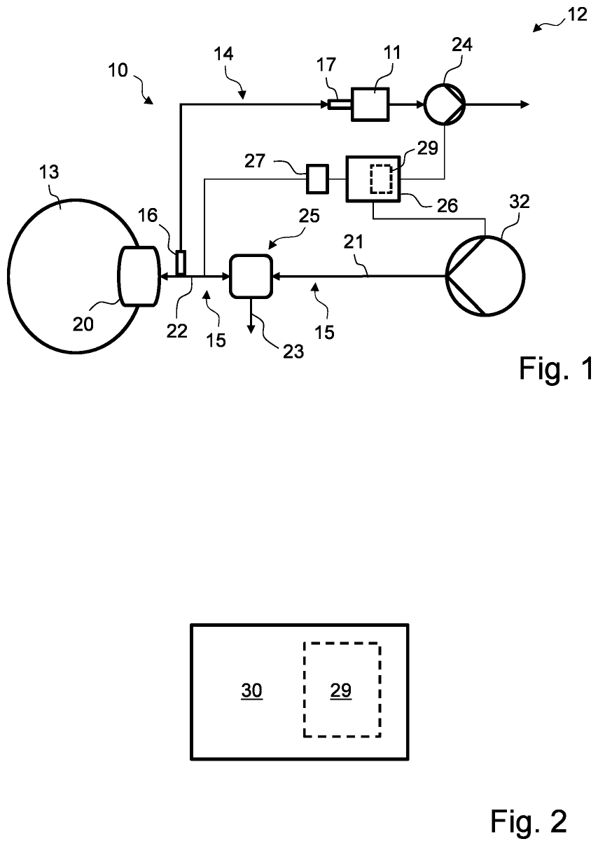

FIG. 1 shows a medical device 12 in the form of a ventilator for ventilating a person 13 according to a first embodiment. The medical device 12 comprises a breathing mask 20 and a main line 15 for sending inhalation gas to the breathing mask 20 and for sending exhalation gas away from the breathing mask 20. The main line 15 has an inhalation gas line section 21 and an exhalation gas line section 23. A main pump 32 is arranged in the inhalation gas line section 21 for feeding inhalation gas to the breathing mask 20 or to the person 13. An exhalation valve 25 is arranged downstream of the main pump 32, when viewed in a flow direction of the inhalation gas. Only inhalation gas is sent in the inhalation gas line section 21 upstream of the exhalation valve 25 and downstream of the main pump 32. Inhalation gas is sent in the exhalation valve 25, through which the main line 15 extends as well, to the breathing mask 20 and exhalation gas is sent away from the breathing mask 20 and into the area surrounding the medical device 12 via the exhalation valve 25. A total gas line section 22, in which inhalation gas is sent during the inhalation phase and exhalation gas is sent during the exhalation phase, is arranged between the exhalation valve 25 and the breathing mask 20.

The medical device 12 shown has, further, a determination device 10 for determining the carbon dioxide concentration in measured gas from the medical device 12 or from the main line during a ventilation of the person 13 by means of the medical device 12. The determination device 10 has a branch line 14 with a first heat and moisture exchanger (heat and moisture exchanger filter) 16 and with a second heat and moisture exchanger 17 for filtering the branched-off measured gas. The determination device 10 has, further, a sensor unit 11 for determining a concentration of carbon dioxide in the measured gas. The branch line 14 is configured for branching off the measured gas from the main line 15 of the medical device 12 during an inhalation phase of the person 13 as inhalation gas and during an exhalation phase of the person 13 as exhalation gas to the sensor unit 11. The first heat and moisture exchanger 16 is arranged directly at the total gas line section 22 with a view to a flow direction of the branched-off and suctioned-off measured gas upstream of the sensor unit 11 and the second heat and moisture exchanger 17 is arranged directly at the sensor unit 11 with a view to a flow direction of the branched-off and suctioned-off measured gas upstream of the sensor unit 11. The two heat and moisture exchangers 16, 17 have each a cylindrical configuration and have each a length of 13 mm and a diameter of 3 mm.

The determination device 10 has a fluid delivery unit 24 in the form of a piezo pump for suctioning off the measured gas from the main line 15 or from the total gas line section 22. The fluid delivery unit 24 is arranged downstream of the sensor unit 11. The heat and moisture exchangers 16, 17 shown have each a microporous plastic foam for filtering the measured gas and for achieving the desired buffering and compensation function concerning the temperature and moisture differences occurring in the measured gas.

Especially the heat conductivity of the exhalation gas is measured in the sensor unit 11 for determining a carbon dioxide concentration in the measured gas. The measurement is carried out by a micro structured heating element on a thin membrane of the sensor unit. A thermophilic unit, which measures an excess temperature of the gas close to the heating element in reference to a silicone frame of the membrane, is located next to the heating element. Additional details about this can be found in German Patent Application DE 10 2010 047 159 A1.

The medical device 12 or the determination device 10 has, furthermore, a setting unit 26, which is configured and embodied for the adaptive setting of the fluid delivery unit 24 taking an airway pressure in the main line 15 into consideration for generating an as uniform as possible volume flow and an as uniform as possible gas pressure of the measured gas in the branch line 14 to the sensor unit 11 during the inhalation phase and during the exhalation phase. The setting unit 25 may be considered to be a control device of the medical device 12. The setting unit 26 is in signal connection with the main pump 32 and with the fluid delivery unit 24 for the setting or actuation of same. The determination device 10 has, in addition, an airway pressure sensor 27 for measuring the airway pressure in the main line 15, wherein the setting unit 26 is configured and embodied for the adaptive setting of the fluid delivery unit 24 taking into consideration the airway pressure in the main line 15, which airway pressure was measured by means of the airway pressure sensor 27, for generating the as uniform as possible volume flow as well as the as uniform as possible gas pressure of the measured gas in the branch line 14 to the sensor unit 11.

A computer program product 29, which comprises commands, which during the execution of the computer program product 29 by the setting unit 26 cause this computer program product to carry out the process described with reference to FIG. 4, is installed in the setting unit 26. A storage device 30, on which such a computer program product 29 is stored, is shown in FIG. 2.

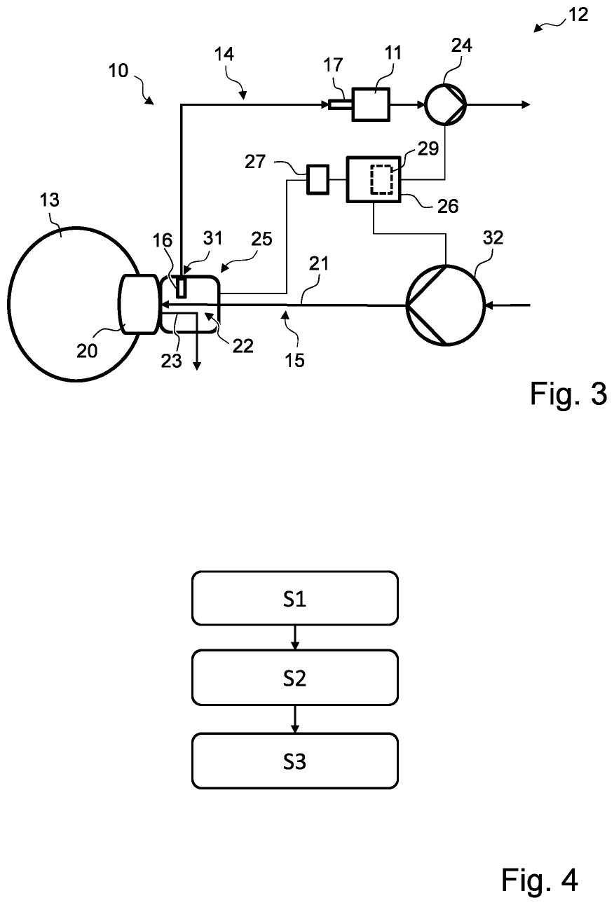

FIG. 3 shows a medical device 12 according to a second embodiment. In the medical device 12 shown in FIG. 3, the exhalation valve 25 is attached to the breathing mask 20. The exhalation valve 25 shown in FIG. 3 has, furthermore, a first heat and moisture exchanger 16, which is integrated into the exhalation valve 25. The first heat and moisture exchanger 16 is arranged according to FIG. 3 directly at a hose line of the branch line 14. The branch line 14 is thus connected to the exhalation valve 25 by means of the hose line and forms a fluid connection to the first heat and moisture exchanger 16 there or makes possible a fluid connection from the main line 15 through the first heat and moisture exchanger 16 to the sensor unit 11. For this, the exhalation valve 25 has a ventil port 31 in the form of a Luer lock fitting for connecting the branch line 14 or the hose line.

FIG. 4 shows a flow chart for explaining a process for determining a concentration of carbon dioxide in measured gas. In a first step S1, the measured gas is first branched off to the sensor unit 11 through the branch line 14 from the main line 15 of a medical device 12 as described above during an inhalation phase of a person 13 connected to the medical device 12 as inhalation gas and during an exhalation phase of the person 13 as exhalation gas. The measured gas is delivered or suctioned off in this case by means of the fluid delivery unit 24 from the main line 15 through the branch line 14 to the sensor unit 11. In a second step S2, which is partly carried out simultaneously with the first step S1, the fluid delivery unit 24, taking the airway pressure in the main line 15 into consideration, is adaptively set for generating an as uniform as possible volume flow as well as an as uniform as possible gas pressure of the measured gas in the branch line 14 to the sensor 11 during the inhalation phase and during the exhalation phase. The airway pressure in the main line 15 is thereby measured by means of an airway pressure sensor 27 and the fluid delivery unit 24 is adaptively set for generating the uniform volume flow and the uniform gas pressure of the measured gas in the branch line 14 to the sensor unit 11, using the measured airway pressure in the main line 15. In the meantime, in a third step S3, the concentration of carbon dioxide in the measured gas is determined by means of the sensor unit 11. More precisely, the heat conductivity of the measured gas is measured by means of the sensor unit 11 to determine the carbon dioxide concentration based thereon as described above.

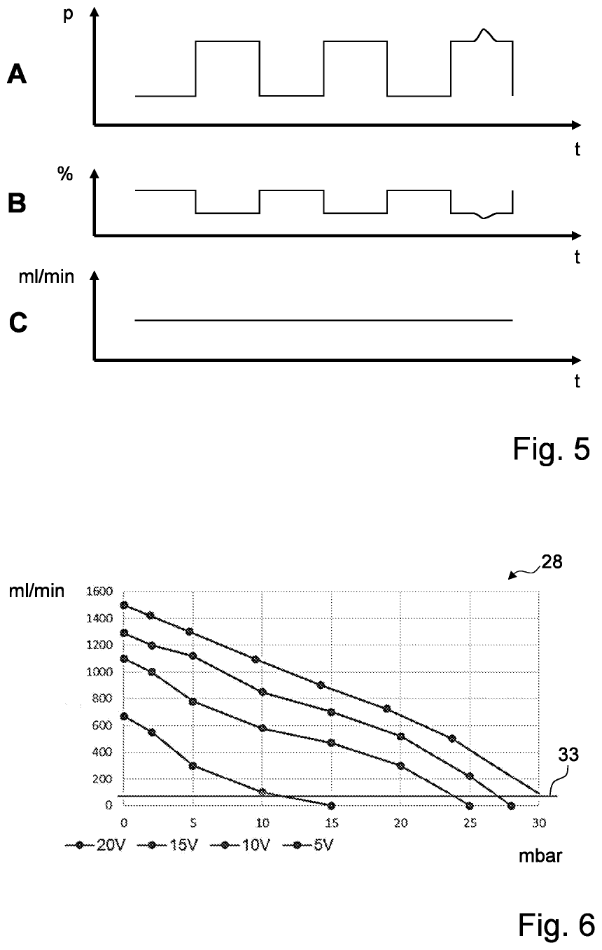

FIG. 5 shows a characteristic diagram for showing an airway pressure during a ventilation with uniform volume flow due to an actuation of the fluid delivery unit 24 as described above. More precisely, diagram A shows a typical curve of the airway pressure. Diagram B shows how the fluid delivery unit 24 is actuated or set in order to generate the as uniform as possible volume flow shown in diagram C.

FIG. 6 shows a look-up table 28, with the use of which the as uniform as possible volume flow as well as the as uniform as possible gas pressure of the measured gas in the branch line 14 to the sensor unit 11 are generated. If a straight line 33 is drawn at 50 mL/min in the look-up table 28, it is seen at first glance what gas pressure results at what operating voltage of the fluid delivery unit 24. This operation can be implemented in an automated manner by means of the setting unit 26 into the proposed process.

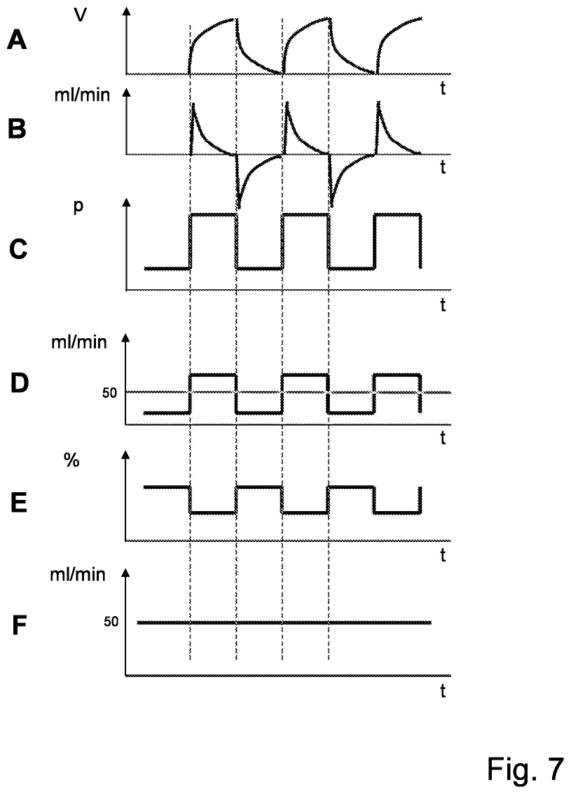

FIG. 7 shows characteristic diagrams for explaining an adaptive setting of a fluid delivery unit 24 during a pressure-controlled ventilation by means of the medical device 12. The volume is plotted over time in characteristic diagram A. The associated volume flow is plotted over time in characteristic diagram B. The associated pressure is plotted over time in characteristic diagram C. Characteristic diagram D shows a changing volume flow for the case, in which the fluid delivery unit 24 is operated uniformly. Characteristic diagram E shows the changing operating output of the fluid delivery unit 24 over time, which results from the above-described, adaptive setting of the fluid delivery unit 24. Characteristic diagram F shows the uniform volume flow, which is generated by the adaptive setting of the fluid delivery unit 24.

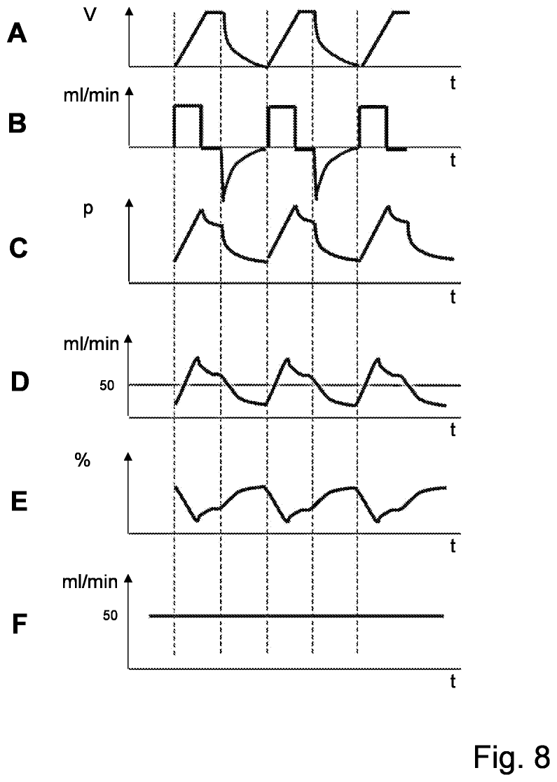

FIG. 8 shows characteristic diagrams for explaining an adaptive setting of a fluid delivery unit during a volume-controlled ventilation by means of a medical device according to the present invention. The volume is plotted over time in characteristic diagram A. The associated volume flow is plotted over time in characteristic diagram B. The associated pressure is plotted over time in characteristic diagram C. Characteristic diagram D shows a changing volume flow for the case, in which the fluid delivery unit 24 is operated uniformly. Characteristic diagram E shows the changing operating output of the fluid delivery unit 24 over time, which results from the above-described, adaptive setting of the fluid delivery unit 24. Characteristic diagram F shows the uniform volume flow, which is generated by the adaptive setting of the fluid delivery unit 24. To determine the concentration of carbon dioxide in the measured gas during the volume-controlled ventilation of the person by means of the medical device 12, the fluid delivery unit 24 is adaptively set for generating the uniform volume flow in the branch line 14 to the sensor unit 11, using in this case the airway pressure in the main line 15, which airway pressure results from the volume-controlled ventilation of the person 13.

The present invention allows additional configuration principles in addition to the embodiments shown. In other words, the present invention shall not be considered to be limited to the exemplary embodiments explained with reference to the figures. In case of a process described above, the fluid delivery unit 24 can thus be adaptively set for generating a uniform volume flow and/or a uniform gas pressure of the measured gas in the branch line 14 to the sensor unit 11, only taking into consideration an airway pressure during an inhalation phase of the ventilation in the main line 15 or only taking into consideration an airway pressure during an exhalation phase of the ventilation in the main line 15. In addition, in a process described above, the fluid delivery unit 24 can be adaptively set for generating a uniform volume flow and/or a uniform gas pressure of the measured gas in the branch line 14 to the sensor unit 11 and it is operated with consistent output during an exhalation phase of the ventilation and during an inhalation phase taking into consideration the airway pressure during the inhalation phase of the ventilation. In addition, the fluid delivery unit 24 can be adaptively set for generating a uniform volume flow and/or a uniform gas pressure of the measured gas in the branch line 14 to the sensor unit 11 during an exhalation phase of the ventilation taking into consideration the airway pressure during the exhalation phase of the ventilation and can be deactivated during an inhalation phase of the ventilation.

While specific embodiments of the invention have been shown and described in detail to illustrate the application of the principles of the invention, it will be understood that the invention may be embodied otherwise without departing from such principles.

### LIST OF REFERENCE NUMBERS

10 Determination device
11 Sensor unit
12 Medical device
13 Person
14 Branch line
15 Main line
16 Heat and moisture exchanger
17 Heat and moisture exchanger
20 Breathing mask
21 Inhalation gas line section
22 Total gas line section
23 Exhalation gas line section
24 Fluid delivery unit
25 Exhalation valve
26 Setting unit
27 Airway pressure sensor
28 Look-up table
29 Computer program product
30 Storage device
31 Valve connection
32 Main pump
33 Straight line

1. A process for determining a carbon dioxide concentration in measured gas, having the following the process comprising the steps of:
branching off the measured gas from a main line of a medical device through a branch line to a sensor unit during an inhalation phase of a person who is connected to the medical device as inhalation gas and during an exhalation phase of the person as exhalation gas;
delivering the measured gas from the main line through the branch line to the sensor unit by means of a fluid delivery unit;
adaptively setting the fluid delivery unit based on an airway pressure in the main line for generating a uniform volume flow and/or gas pressure of the measured gas in the branch line to the sensor unit during the inhalation phase and the exhalation phase; and
determining the concentration of carbon dioxide in the measured gas by means of the sensor unit.

2. A process in accordance with claim 1, wherein the airway pressure in the main line is measured by an airway pressure sensor and the fluid delivery unit is adaptively set for generating a uniform volume flow and/or gas pressure of the measured gas in the branch line to the sensor unit, with the use of the measured airway pressure in the main line.

3. A process in accordance with claim 1 for determining the carbon dioxide concentration in the measured gas during a pressure-controlled ventilation of the person by means of the medical device, wherein the fluid delivery unit is adaptively set for generating a uniform volume flow and/or gas pressure of the measured gas in the branch line to the sensor unit, with the use of an airway pressure in the main line, which airway pressure was is set by a pressure-controlled ventilation of the person.

4. A process in accordance with claim 1 for determining the carbon dioxide concentration in the measured gas during a volume-controlled ventilation of the person by means of the medical device, wherein the fluid delivery unit is adaptively set for generating a uniform volume flow and/or gas pressure of the measured gas in the branch line to the sensor unit, with the use of an airway pressure in the main line, which airway pressure results from a volume-controlled ventilation of the person.

5. A process in accordance with claim 1, wherein the fluid delivery unit has a piezo pump for delivering the measured gas from the main line through the branch line to the sensor unit and an operating voltage of the piezo pump is adaptively set for generating a uniform volume flow and/or gas pressure of the measured gas in the branch line to the sensor unit, taking into consideration the airway pressure as well as with the use data of a look-up table.

6. A process in accordance with claim 1, wherein the fluid delivery unit is adaptively set for generating a uniform volume flow and/or gas pressure in the measured gas in the branch line to the sensor unit, only based on an airway pressure during an inhalation phase of the ventilation in the main line or only based on an airway pressure during an exhalation phase of the ventilation in the main line.

7. A process in accordance with claim 1, wherein the fluid delivery unit is operated with consistent output during an exhalation phase of the ventilation for generating a uniform volume flow and/or gas pressure of the measured gas in the branch line to the sensor unit and is adaptively set during an inhalation phase of the ventilation based on the airway pressure during the inhalation phase of the ventilation.

8. A process in accordance with claim 1, wherein the fluid delivery unit is adaptively set for generating a uniform volume flow and/or gas pressure of the measured gas in the branch line to the sensor unit during an exhalation phase of the ventilation based on the airway pressure during the exhalation phase of the ventilation and is deactivated during an inhalation phase of the ventilation.

9. A determination device for determining a carbon dioxide concentration in measured gas from a medical device during a ventilation of a person by means of the medical device, the determination device comprising:
a sensor unit for determining the concentration of carbon dioxide in the measured gas;
a branch line for branching off the measured gas from a main line of the medical device, during an inhalation phase of the person as inhalation gas and during an exhalation phase of the person as exhalation gas, to the sensor unit;
a fluid delivery unit for delivering the measured gas from the main line through the branch line to the sensor unit; and
a setting unit, which is configured and embodied for an adaptive setting of the fluid delivery unit based on an airway pressure in the main line for generating a uniform volume flow and/or gas pressure of the measured gas in the branch line to the sensor unit during the inhalation phase and during the exhalation phase.

10. A determination device in accordance with claim 9, further comprising an airway pressure sensor for measuring the airway pressure in the main line, wherein the setting unit is configured and embodied for the adaptive setting of the fluid delivery unit based on the measured airway pressure in the main line for generating a uniform volume flow and/or gas pressure of the measured gas in the branch line to the sensor unit.

11. A determination device in accordance with claim 9, further comprising at least one heat and moisture exchanger filter configured in and/or at the branch line at least one heat.

12. A determination device in accordance with claim 11, wherein the at least one heat and moisture exchanger filter has a length in a range of 8 mm to 20 mm and a width in a range of 2 mm to 6 mm.

13. A determination device in accordance with claim 9, wherein the fluid delivery unit comprises a piezo pump.

14. A determination device in accordance with claim 9, for determining a carbon dioxide concentration in measured gas from the medical device during a pressure-controlled ventilation of a person by means of the medical device, wherein the setting unit is configured for the adaptive setting of the fluid delivery unit with the use of an airway pressure in the main line, which airway pressure is set by means of a pressure-controlled ventilation of the person with the medical device.

15. A determination device in accordance with claim 9, for determining a carbon dioxide concentration in measured gas from a medical device during a volume-controlled ventilation of a person by means of the medical device, wherein the setting unit is configured for the adaptive setting of the fluid delivery unit with the use of an airway pressure in the main line, which airway pressure results from a volume-controlled ventilation of the person with the medical device.

16. A determination device in accordance with claim 9, in combination with the medical device for ventilating a person, having the main line for sending inhalation gas and for sending exhalation gas.

17. A determination device in combination with the medical device in accordance with claim 16, wherein the main line has an inhalation gas line section for sending an inhalation gas and a total gas line section for sending the inhalation gas as well as an exhalation gas, wherein the branch line is configured for branching off the measured gas from the total gas line section.

18. A determination device in combination with the medical device in accordance with claim 17, wherein an exhalation valve in the total gas line section is configured for releasing the exhalation gas from the medical device into the area surrounding the medical device, wherein at least one heat and moisture exchanger filter is provided in the exhalation valve.

19. A determination device in combination with the medical device in accordance with claim 18, wherein the branch line for branching off the measured gas from the main line is connected to the exhalation valve.

20. A determination device in combination with the medical device in accordance with claim 16, wherein the medical device is configured as a ventilator.

21. A setting unit for use in a determination device in accordance with claim 9, wherein the setting device which is configured and embodied for the adaptive setting of the fluid delivery unit based on the airway pressure in the main line for generating a uniform volume flow and/or gas pressure in the measured gas in the branch line to the sensor unit during the inhalation phase and/or during the exhalation phase.

22. A process according to claim 1, wherein a non-transitory computer-readable media computer program product, comprising commands, which during the execution of the computer program product by a computer cause this computer program product to carry out at least some of the process steps.

23. A non-transitory computer-readable media storage device comprising a non-transitory computer-readable media computer program product stored thereon, the non-transitory computer-readable media computer program product comprising commands, which during the execution of the computer program product by a computer cause this computer program product to carry out the process comprising the steps of:
with a branching off the measured gas from a main line of a medical device through a branch line to a sensor unit during an inhalation phase of a person who is connected to the medical device as inhalation gas and during an exhalation phase of the person as exhalation gas delivering the measured gas from the main line through the branch line to the sensor unit by means of a fluid delivery unit by adaptively setting the fluid delivery unit based on an airway pressure in the main line for generating a uniform volume flow and/or gas pressure of the measured gas in the branch line to the sensor unit during the inhalation phase and the exhalation phase; and
determining the concentration of carbon dioxide in the measured gas by means of the sensor unit.
